SEQUENCE LISTING (11 sequences)

SEQ ID NO: 1 - DNA, 37 bp, Artificial Sequence
  SYNTHESIZED
atcttgcctgatttgtaaatacaaagttgactgtgaa

SEQ ID NO: 2 - DNA, 37 bp, Artificial Sequence
  SYNTHESIZED
ggctcctactctgtttgcaagcgatgcatacatgcaa

SEQ ID NO: 3 - DNA, 30 bp, Artificial Sequence
  SYNTHESIZED
accccacagtggggccactagggacaggat

SEQ ID NO: 4 - protein, 7 aa, Artificial Sequence
  SYNTHESIZED
PKKKRKV

SEQ ID NO: 5 - protein, 7 aa, Artificial Sequence
  SYNTHESIZED
PKKKRRV

SEQ ID NO: 6 - protein, 16 aa, Artificial Sequence
  SYNTHESIZED
KRPAATKKAGQAKKKK

SEQ ID NO: 7 - protein, 20 aa, Artificial Sequence
  SYNTHESIZED
GRKKRRQRRRPPQPKKKRKV

SEQ ID NO: 8 - protein, 19 aa, Artificial Sequence
  SYNTHESIZED
PLSSIFSRIGDPPKKKRKV

SEQ ID NO: 9 - protein, 24 aa, Artificial Sequence
  SYNTHESIZED
GALFLGWLGAAGSTMGAPKKKRKV

SEQ ID NO: 10 - protein, 27 aa, Artificial Sequence
  SYNTHESIZED
GALFLGFLGAAGSTMGAWSQPKKKRKV

SEQ ID NO: 11 - protein, 21 aa, Artificial Sequence
  SYNTHESIZED
KETWWETWWTEWSQPKKKRKV

(19) United States
(12) Patent Application Publication
(10) Pub. No.: US 2016/0145645 A1
(43) Pub. Date: May 26, 2016

(54) TARGETED INTEGRATION

(71) Applicant: SIGMA-ALDRICH CO. LLC, St, Louis, MO (US)

(72) Inventors: Scott BAHR, St. Louis, MO (US); Trissa BORGSCHULTE, St. Louis, MO (US); Kevin KAYSER, St. Louis, MO (US)

(73) Assignee: Sigma-Aldrich Co. LLC, St. Louis, MO (US)

(21) Appl. No.: 14/899,405

(22) PCT Filed: Jun. 19, 2014
(86) PCT No.: PCT/US2014/043138

§ 371 Date: Dec. 17, 2015

Related U.S. Application Data
(60) Provisional application No. 61/837,019, filed on Jun. 19, 2013.

(51) Int. Cl. C12N 15/90; C12P 21/00

(52) U.S. Cl. CPC C12N 15/907; C12P 21/00

(57) ABSTRACT

The present disclosure encompasses an isolated cell comprising an exogenous nucleic acid sequence located within or proximal to a predetermined genomic locus, wherein the exogenous nucleic acid sequence comprises at least one recognition sequence which can be exploited by one or more polynucleotide modification enzymes for targeted integration of a recombinant protein. The disclosure further provides methods for preparing such cells, and methods for retargeting such cells for the production of recombinant proteins, and kits for the same.

### CROSS-REFERENCE TO RELATED APPLICATIONS

This application is a U.S. National Stage Application of PCT International Application No. PCT/US2014/043138, filed Jun. 19, 2014, which claims priority to U.S. Provisional Application Ser. No. 61/837,019, filed Jun. 19, 2013, the disclosure of each is hereby incorporated by reference in its entirety.

### FIELD

The present disclosure relates to the targeted integration of sequences encoding recombinant proteins into cells of interest. In particular, a cell of interest comprises an exogenous nucleic acid sequence located within or proximal to a predetermined genomic locus, wherein the exogenous nucleic acid sequence comprises at least one recognition sequence which can be exploited by one or more polynucleotide modification enzymes for targeted integration of the sequence encoding the recombinant protein.

### BACKGROUND

In recent years, targeted integration (TI) of recombinant protein expression constructs at defined locations within the genomes of mammalian cells has sparked much interest in the biopharmaceutical industry. TI technologies allow cell line development scientists to integrate transgenes of interest into predefined, well characterized genomic loci, thereby enabling the prediction of recombinant protein expression characteristics which may lead to increased cell line stability, decreased clone-to-clone and molecule-to-molecule heterogeneity and overall decreased cell line development timelines. Chinese Hamster Ovary (CHO) cells are the most commonly used cell line for the production of biotherapeutic proteins. However, despite their recognized usefulness in therapeutic protein production, to date, TI in CHO cells has been met with limited success. Accordingly, improved methods of executing TI in CHO and other cells are needed that would benefit the bioproduction industry.

### SUMMARY

Among the various aspects of the present disclosure is the provision of an isolated cell comprising at least one exogenous nucleic acid sequence located in genomic DNA within or proximal to at least one genomic locus listed in Table 2, wherein each exogenous nucleic acid sequence comprises at least one recognition sequence for a polynucleotide modification enzyme. In one embodiment, the cell is a CHO cell. In another embodiment, the at least one recognition sequence comprises a nucleic acid sequence that does not exist endogenously in the genome of the cell (or CHO cell). In a further embodiment, the polynucleotide modification enzyme is a targeting endonuclease (e.g., zinc finger nuclease (ZFN), meganuclease, transcription activator-like effector nuclease (TALEN), CRIPSR endonuclease, I-Tevl nuclease or related monomeric hybrids, or artificial targeted DNA double strand break inducing agent), a site-specific recombinase (e.g., lambda integrase, Cre recombinase, FLP recombinase, gamma-delta resolvase, Tn3 resolvase, ΦC31 integrase, Bxb1-integrase, or R4 integrase), or combinations thereof. In a further embodiment, a first recognition sequence is recognized by a first ZFN pair. In still another embodiment, a first recognition sequence is recognized by a first ZFN pair and a second recognition sequence is recognized by a second ZFN pair that differs from the first pair of ZFN. In one iteration, the first and the second ZFN pair are selected from the group consisting of hSIRT, hRSK4, and hAAVS1. In still another embodiment, the exogenous nucleic acid sequence further comprises at least one selectable marker sequence, at least one reporter sequence, at least one regulatory control sequence element, or combinations thereof.

Another aspect of the present disclosure encompasses a method for preparing a cell comprising at least one exogenous nucleic acid sequence comprising at least one recognition sequence for a polynucleotide modification enzyme. The method comprises (a) introducing into a cell at least one targeting endonuclease that is targeted to a sequence within or proximal to a genomic locus listed in Table 2; (b) introducing into the cell at least one donor polynucleotide comprising the exogenous nucleic acid that is flanked by (i) sequences having substantial sequence identity to the targeted genomic locus or (ii) the recognition sequence of the targeting endonuclease; and (c) maintaining the cell under conditions such that the exogenous nucleic acid is integrated into genome of the cell. In one embodiment, the cell is a CHO cell. In another embodiment, the exogenous nucleic acid is integrated into the genome by a homology-directed process. In a further embodiment, the exogenous nucleic acid is integrated into the genome by a direct ligation process. In still another embodiment, the targeting endonuclease is selected from the group consisting of zinc finger nuclease (ZFN), meganuclease, transcription activator-like effector nuclease (TALEN), CRIPSR endonuclease, I-Tevl nuclease or related monomeric hybrids, and artificial targeted DNA double strand break inducing agent.

A further aspect of the present disclosure provides a method for retargeting a cell for the production of at least one recombinant protein. The method comprises (a) providing a cell comprising at least one exogenous recognition sequence for a polynucleotide modification enzyme located within or proximal to at least one genomic locus listed in Table 2; (b) introducing into the cell (i) at least one expression construct comprising a sequence encoding a recombinant protein that is flanked by first and second sequences, and (ii) at least one polynucleotide modification enzyme that recognizes the at least one exogenous recognition sequence in the cell; and (c) maintaining the cell under conditions such that the sequence encoding the recombinant protein is integrated into the genome of the cell. In one embodiment, the cell is a CHO cell. In another embodiment, the at least one exogenous recognition sequence of the cell is a targeting endonuclease recognition site; the first and second sequences of the expression construct are sequences with substantial sequence identity to chromosomal sequence near the exogenous recognition sequence in the cell; and the at least one polynucleotide modification enzyme is a targeting endonuclease. In still another embodiment, the at least one exogenous recognition sequence of the cell is a targeting endonuclease recognition site; each of the first and second sequences of the expression construct is the recognition sequence of the targeting endonuclease; and the at least one polynucleotide modification enzyme is a targeting endonuclease. In some embodiments, the targeting endonuclease is a zinc finger nuclease (ZFN), a meganuclease, a transcription activator-like effector nuclease (TALEN), a CRIPSR endonuclease, an I-Tevl nuclease or related monomeric hybrids, or an artificial targeted DNA double strand break inducing agent. In a further embodiment, the at least one exogenous recognition sequence of the cell is a site-specific recombinase recognition site; each of the first and second sequences of the expression construct is the site-specific recombinase recognition sequence; and the at least one polynucleotide modification enzyme is a site-specific recombinase, wherein the site-specific recombinase is selected from the group consisting of lambda integrase, Cre recombinase, FLP recombinase, gamma-delta resolvase, Tn3 resolvase, ΦC31 integrase, Bxb1-integrase, and R4 integrase. In an additional embodiment, the sequence encoding a recombinant protein is operably linked to at least one expression control sequence. In an alternate embodiment, the expression construct further comprises at least one selectable marker sequence, at least one reporter sequence, at least one regulatory control sequence element, or combinations thereof. In yet another embodiment, the cells are maintained under conditions for expression of the at least one recombinant protein.

Still another aspect of the present disclosure encompasses a kit for retargeting a cell for the production of a recombinant protein. The kit comprises a cell comprising at least one exogenous nucleic acid sequence located in genomic DNA within or proximal to at least one genomic locus listed in Table 2, wherein each exogenous nucleic acid sequence comprises at least one recognition sequence for a polynucleotide modification enzyme, along with a polynucleotide modification enzyme corresponding to the recognition sequence and an construct for insertion of sequence encoding the recombinant protein of interest, wherein the construct further comprises a pair of flanking sequences corresponding to the recognition sequence and/or the genomic DNA flanking the recognition sequence. In one embodiment, the cell is a CHO cell. In another embodiment, the kit further comprises instructions for completing targeted integration of the sequence encoding the recombinant protein. In some embodiments, the polynucleotide modification enzyme is a targeting endonuclease selected from the group consisting of zinc finger nuclease (ZFN), meganuclease, transcription activator-like effector nuclease (TALEN), CRIPSR endonuclease, I-Tevl nuclease or related monomeric hybrids, and artificial targeted DNA double strand break inducing agent. In other embodiments, the polynucleotide modification enzyme is a site-specific recombinase selected from the group consisting of lambda integrase, Cre recombinase, FLP recombinase, gamma-delta resolvase, Tn3 resolvase, ΦC31 integrase, Bxb1-integrase, and R4 integrase.

Additional aspects and iterations of the disclosure are detailed below.

### BRIEF DESCRIPTION OF THE FIGURES

FIG. 1 is a schematic representation of a donor plasmid used for integration of the human AAVS1 ZFN recognition sequence into the CHO genomic location Refseq. ID NW 003618207.1, base pairs 5366-20679.

FIG. 2 is a schematic representation of Refseq. ID NW 003618207.1, base pairs 5366-20679 containing the integrated AAVS landing pad. The primer binding sites used for the junction PCR are indicated.

FIG. 3A shows a schematic representation of a donor that can be used to introduce recombinant protein expression constructs into a genome by ZFN mediated targeted integration. The desired sequence to be integrated, comprising, for example, the recombinant protein expression construct(s), (referred to herein as the “payload” sequence) is flanked by sequences (i.e., homology arms) that are homologous to the genomic DNA sequences surrounding the ZFN recognition sequence. This design will allow for targeted integration via classical homologous recombination. The payload may include an expression cassette for the recombinant protein of interest along with an expression cassette for a selectable marker. Other elements in the payload could include reporters, promoters, or any other exogenous sequence. FIG. 3B shows an alternate donor that can be used to introduce recombinant protein expression constructs into a genome by ZFN mediated targeted integration. The payload is flanked by the same ZFN recognition sequence (ZFN RS) as that being targeted in the host cell genome. Therefore upon transfection with the ZFN pair, the ZFNs will cut both the endogenous genomic DNA as well as the donor DNA, leaving sticky cohesive ends that will allow for the targeted integration of the payload via DNA repair mechanisms. The payload may include an expression cassette for the recombinant protein of interest along with an expression cassette for a selectable marker. Other elements in the payload could include reporters, promoters, or any other exogenous sequence.

### DETAILED DESCRIPTION

Targeted integration of sequences encoding recombinant proteins, particularly biotherapeutic protein products, is strongly preferred over random integration, both for the efficiency of incorporation of the desired genetic material, and also for the improved stability, homogeneity, and level of protein expression following integration. Endonuclease technologies, such as zinc finger nuclease (ZFN) technology as well as other technologies discussed herein, now allow the introduction of site-specific modification of endogenous genomic sequences, with greater efficiency and opportunity for customization than with certain prior methods of targeted integration. The present disclosure provides cells useful for targeted integration of sequences encoding recombinant proteins, which cells are particularly suitable due to incorporation of a “landing pad” site in their genome. Chinese Hamster Ovary (CHO) or other mammalian cells may be modified as described herein to receive such landing pad, i.e., modified to include a synthetic nucleotide sequence comprising one or more recognition sequences for a polynucleotide modification enzyme such as a site-specific recombinase and/or a targeting endonuclease. The landing pad may be inserted at a suitable locus for expression of the recombinant protein(s). Following integration of the landing pad (sequence comprising one or more recognition sequences for a polynucleotide modification enzyme) at a particular position within the genome, sequence encoding one or more proteins may be inserted at the location containing the one or more recognition sequences using a corresponding recombinase and/or targeted endonuclease, with such insertion occurring at higher levels of efficiency than with random integration or other previously described methods. It will be understood that multiple landing pads can be located at different positions in the genome, allowing for multi-copy integration of recombinant protein expression constructs or cassettes as well as multiple unique protein expression cassettes.

### I. Exogenous Sequence Comprising at Least One Recognition Sequence

In one aspect, the present disclosure encompasses an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tevl nucleases or related monomeric hybrids, or artificial targeted DNA double strand break inducing agents. Each of these targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases. Three exemplary ZFN recognition sequences are provided at Table 1, below.

TABLE 1
ZFN Recognition Sequences

| | | | SEQ. |
|---|---|---|---|
| ZFN | | ZFN | ID. |
| Pair | Sequence (5′-3′) | Activity | NO: |
| hSIRT | ATCTTGCCTGATTTGTaaa | 16.7% | 1 |
| | tacAAAGTTGACTGTGAA | | |
| hRSK4 | GGCTCCTACTCTGTTTgca | 65.7% | 2 |
| | agcGATGCATACATGCAA | | |
| hAAVS1 | ACCCCACAGTGGggccacT | 27.6% | 3 |
| | AGGGACAGGAT | | |

Multiple recognition sequences may be present in a single landing pad, allowing the landing pad to be targeted sequentially by two or more polynucleotide modification enzymes such that two or more unique payload sequences (comprising, among other things, protein expression cassettes) can be inserted. Alternatively, the presence of multiple recognition sequences in the landing pad, allows multiple copies of the same payload sequence to be inserted into the landing pad. When two payload sequences are targeted to a single landing pad, the landing pad includes a first recognition sequence for a first polynucleotide modification enzyme (such as a first ZFN pair), and a second recognition sequence for a second polynucleotide enzyme (such as a second ZFN pair). Alternatively, or additionally, individual landing pads comprising one or more recognition sequences may be integrated at multiple locations within a cell's genome to permit multi-copy integration of payload sequences comprising recombinant protein expression constructs. Increased protein expression may be observed in cells transformed with multiple copies of a payload sequence comprising an expression construct. Alternatively, multiple protein products may be expressed simultaneously when multiple unique payload sequences comprising different expression cassettes are inserted, whether in the same or a different landing pad. Regardless of the number and type of payload sequences, when the targeting endonuclease is a ZFN, exemplary ZFN pairs include hSIRT, hRSK4, and hAAVS1, with accompanying recognition sequences as identified in Table 1, above.

Generally speaking, an exogenous nucleic acid used as a landing pad may comprise at least one recognition sequence. For example, an exogenous nucleic acid may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable markers such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named LAGLIDADG has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

(a) Zinc Finger Nucleases

A non-limiting, exemplary targeting endonuclease is a zinc finger nuclease (ZFN). Typically, a zinc finger nuclease comprises a DNA binding domain (i.e., zinc finger) and a cleavage domain (i.e., nuclease), both of which are described below.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain can have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm described in U.S. Pat. No. 6,453,242 may be used to design a zinc finger binding domain to target a preselected sequence. Alternative methods, such as rational design using a nondegenerate recognition code table can also be used to design a zinc finger binding domain to target a specific sequence (Sera et al. (2002) Biochemistry 41:7074-7081). Publically available web-based tools for identifying potential target sites in DNA sequences and designing zinc finger binding domains are found at www.zincfingertools.org and zifit.partners.org/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize and bind a DNA sequence ranging from about 3 nucleotides to about 21 nucleotides in length, for example, from about 9 to about 18 nucleotides in length. Each zinc finger recognition region (i.e., zinc finger) recognizes and binds three nucleotides. In general, the zinc finger binding domains of the zinc finger nucleases disclosed herein comprise at least three zinc finger recognition regions (i.e., zinc fingers). The zinc finger binding domain may for example comprise four zinc finger recognition regions. Alternatively, the zinc finger binding domain may comprise five or six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227, the disclosure of which is incorporated herein by reference.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers (and additional domains) of the protein.

(ii) Cleavage Domain

A zinc finger nuclease also includes a cleavage domain. The cleavage domain portion of the zinc finger nuclease may be obtained from any endonuclease or exonuclease. Non-limiting examples of endonucleases from which a cleavage domain may be derived include, but are not limited to, restriction endonucleases and homing endonucleases. See, for example, New England Biolabs catalog (www.neb.com) and Belfort et al. (1997) Nucleic Acids Res. 25:3379-3388. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease can comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is FokI. This particular enzyme is active as a dimer (Bitinaite et al. (1998) Proc. Natl. Acad. Sci. USA 95: 10, 570-10, 575). Accordingly, for the purposes of the present disclosure, the portion of the FokI enzyme used in a zinc finger nuclease is considered a cleavage monomer. Thus, for targeted double-stranded cleavage using a FokI cleavage domain, two zinc finger nucleases, each comprising a FokI cleavage monomer, may be used to reconstitute an active enzyme dimer. Alternatively, a single polypeptide molecule containing a zinc finger binding domain and two FokI cleavage monomers can also be used.

The cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains. Exemplary engineered cleavage monomers of FokI that form obligate heterodimers include a pair in which a first cleavage monomer includes mutations at amino acid residue positions 490 and 538 of FokI and a second cleavage monomer that includes mutations at amino-acid residue positions 486 and 499 (Miller et al., 2007, Nat. Biotechnol, 25:778-785; Szczpek et al., 2007, Nat. Biotechnol, 25:786-793). For example, the Glu (E) at position 490 may be changed to Lys (K) and the Ile (I) at position 538 may be changed to K in one domain (E490K, I538K), and the Gln (Q) at position 486 may be changed to E and the I at position 499 may be changed to Leu (L) in another cleavage domain (Q486E, I499L). In other aspects, modified FokI cleavage domains can include three amino acid changes (Doyon et al. 2011, Nat. Methods, 8:74-81). For example, one modified FokI domain (which is termed ELD) can comprise Q486E, I499L, N496D mutations and the other modified FokI domain (which is termed KKR) can comprise E490K, I538K, H537R mutations.

(iii) Additional Domains

In some aspects, the zinc finger nuclease further comprises at least one nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027. The NLS may be located at the N-terminus, the C-terminal, or in an internal location of the zinc finger nuclease.

In other aspects, the zinc finger nuclease may also comprise at least one cell-penetrating domain. The cell-penetrating domain may be a cell-penetrating peptide sequence derived from the HIV-1 TAT protein, a cell-penetrating peptide sequence derived from the human hepatitis B virus, a cell penetrating peptide from Herpes simplex virus, MPG peptide, Pep-1 peptide, or a polyarginine peptide sequence. The cell-penetrating domain may be located at the N-terminus, the C-terminal, or in an internal location of the zinc finger nuclease.

(b) RNA-Guided Endonucleases

The RNA-guided endonuclease may be derived from a clustered regularly interspersed short palindromic repeats (CRISPR)/CRISPR-associated (Cas) system. The CRISPR/Cas system may be a type I, a type II, or a type III system. In some aspects, the RNA-guided endonuclease may be derived from a type II CRISPR/Cas system. The type II system may be a Csn1 subfamily or a Csx12 subfamily. In exemplary aspects, the endonuclease may be derived from a Cas9 protein of a type II system. In various aspects, the endonuclease may be derived from a Cas9 protein (or Cas9 homolog) from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, and so forth. In exemplary aspects, the endonuclease is derived from a Cas9 protein from a Streptococcus species.

The RNA-guided endonuclease may be derived from a wild type Cas9 protein or fragment thereof. In other aspects, the RNA-guided endonuclease may be derived from modified Cas9 protein. For example, the amino acid sequence of the Cas9 protein may be modified such that one or more properties (e.g., nuclease activity, affinity, stability, etc.) of the protein is improved. Alternatively, domains of the Cas9 protein not involved in RNA-guided cleavage may be eliminated from the protein such that the modified Cas9 protein is smaller than the wild type Cas9 protein. In still other aspects, the RNA-guided endonuclease may be a fusion protein comprising domains of wild type Cas9 proteins, modified Cas9 proteins, and/or other proteins. For example the RNA-guided endonuclease could comprise a marker, such as GFP or another fluorescent protein.

In general, a Cas9 protein comprises a RuvC-like nuclease domain and a HNH-like nuclease domain. In some aspects, the Cas9-derived endonuclease can comprise two functional nuclease domains, e.g., a RuvC-like nuclease domain and a HNH-like nuclease domain. In such aspects, the endonuclease can cleave a double-stranded nucleic acid. In other aspects, the Cas9-derived endonuclease can comprise only one functional nuclease domain (either a RuvC-like or a HNH-like nuclease domain). In these aspects, the endonuclease can cleave a single-stranded nucleic acid or introduce a nick into a double-stranded nucleic acid. The nuclease domains of the RNA-guided endonuclease may be derived from the same Cas9 protein or they may be derived from different Cas9 proteins.

The Cas9-derived endonucleases disclosed herein comprise at least one nuclear localization signal (NLS) for transport into the nuclei of eukaryotic cells. In general, an NLS comprise a stretch of basic amino acids. Nuclear localization signals are known in the art (see, e.g., Lange et al., J. Biol. Chem., 2007, 282:5101-5105). For example, in one embodiment, the NLS may be monopartite sequence such as PKKKRKV (SEQ ID NO:4) or PKKKRRV (SEQ ID NO:5). In another embodiment, the NLS may be a bipartite sequence. In still another embodiment, the NLS may be KRPAATKKAGQAKKKK (SEQ ID NO:6). The NLS may be located at the N-terminus, the C-terminal, or in an internal location of the endonuclease. In a non-limiting example, the NLS is located at the C-terminus of the endonuclease.

In general, the RNA-guided endonuclease is a DNA endonuclease. In some aspects, the RNA-guided endonuclease can cleave one strand of double-stranded DNA. In exemplary aspects, the RNA-guided endonuclease can cleave both strands of double-stranded DNA. The DNA, for example, may be linear or circular. In exemplary iterations, the DNA is chromosomal (i.e., associated with histones and other chromosomal proteins).

(c) CRISPR/Cas-Like Fusion Proteins

One aspect of the present disclosure provides a fusion protein comprising a CRISPR/Cas-like protein or fragment thereof and an effector domain. These fusion proteins may be used in any of the aspects described above with regard to RNA-guided endonucleases. The CRISPR/Cas-like protein is derived from a clustered regularly interspersed short palindromic repeats (CRISPR)/CRISPR-associated (Cas) system protein. The effector domain may be a cleavage domain, a transcriptional activation domain, a transcriptional repressor domain, or an epigenetic modification domain.

(i) CRISPR/Cas-Like Protein Domain

The fusion protein comprises a CRISPR/Cas-like protein or a fragment thereof. The CRISPR/Cas-like protein may be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the CRISPR/Cas-like protein of the fusion protein is derived from a type II CRISPR/Cas system. In exemplary aspects, the CRISPR/Cas-like protein of the fusion protein is derived from a Cas9 protein. The Cas9 protein may be from any suitable species such as those identified above.

In general, CRISPR/Cas-like proteins comprise at least one RNA recognition and/or RNA binding domain. RNA recognition and/or RNA binding domains interact with the guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein of the fusion protein may be a wild type CRISPR/Cas protein, a modified CRISPR/Cas protein, or a fragment of a wild type or modified CRISPR/Cas protein. The CRISPR/Cas protein may be modified to increase nucleic acid binding affinity and/or specificity, alter an enzymatic activity, and/or change another property of the protein. For example, nuclease (i.e., DNase, RNase) domains of the CRISPR/Cas protein may be modified or inactivated. Alternatively, the CRISPR/Cas protein may be truncated to remove domains that are not essential for the function of the fusion protein. Alternatively, the CRISPR/Cas protein may be truncated or modified to optimize the activity of the effector domain of the fusion protein.

In some aspects, the CRISPR/Cas-like protein of the fusion protein may be derived from a wild type Cas9 protein or fragment thereof. In other aspects, the CRISPR/Cas-like protein of the fusion protein may be derived from modified Cas9 protein. For example, the amino acid sequence of the Cas9 protein may be modified to alter one or more properties (e.g., nuclease activity, affinity, stability, etc.) of the protein. Alternatively, domains of the Cas9 protein not involved in RNA-guided cleavage may be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. In some aspects, the Cas9-derived protein may be modified to contain only one functional nuclease domain (either a RuvC-like or a HNH-like nuclease domain). In these aspects, the Cas9-derived protein is able to introduce a nick into a double-stranded nucleic acid. For example, an aspartate to alanine (D10A) conversion in a RuvC-like domain converts the Cas9-derived protein into a nickase. In other aspects, both of the RuvC-like nuclease domain and the HNH-like nuclease domain may be modified or eliminated such that the Cas9-derived protein is unable to cleave double stranded nucleic acid. In still other aspects, all nuclease domains of the Cas9-derived protein may be modified or eliminated such that the Cas9-derived protein lacks all nuclease activity. The nuclease domains may be inactivated by deletion mutations, insertion mutations, and/or substitution mutations. In a non-limiting example, the CRISPR/Cas-like protein of the fusion protein is derived from a Cas9 protein in which all the nuclease domains have been inactivated or deleted.

The fusion protein also comprises an effector domain. The effector domain may be a cleavage domain or another suitable domain as determined by one of ordinary skill in the art. In preferred aspects of the present disclosure, the effector domain is a cleavage domain. The effector domain may be located at the carboxy or the amino terminal end of the fusion protein.

(ii) Effector Domain

In some aspects, the effector domain is a cleavage domain. As used herein, a “cleavage domain” refers to a domain that cleaves DNA. The cleavage domain may be obtained from any endonuclease or exonuclease. Non-limiting examples of endonucleases from which a cleavage domain may be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

In some aspects, the cleavage domain may be derived from a type II-S endonuclease. Type II-S endonucleases cleave DNA at sites that are typically several base pairs away the recognition site and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In exemplary aspects, the cleavage domain of the fusion protein is a FokI cleavage domain or a derivative thereof.

In certain aspects, the type II-S cleavage may be modified to facilitate dimerization of two different cleavage domains (each of which is attached to a CRISPR/Cas-like protein or fragment thereof). For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI cleavage domains are targets for modification. For example, modified cleavage domains of FokI that form obligate heterodimers include a pair in which a first modified cleavage domain includes mutations at amino acid positions 490 and 538 and a second modified cleavage domain that includes mutations at amino acid positions 486 and 499 (Miller et al., 2007, Nat. Biotechnol, 25:778-785; Szczpek et al., 2007, Nat. Biotechnol, 25:786-793). For example, the Glu (E) at position 490 may be changed to Lys (K) and the Ile (I) at position 538 may be changed to K in one domain (E490K, I538K), and the Gin (Q) at position 486 may be changed to E and the I at position 499 may be changed to Leu (L) in another cleavage domain (Q486E, I499L). In other aspects, modified FokI cleavage domains can include three amino acid changes (Doyon et al. 2011, Nat. Methods, 8:74-81). For example, one modified FokI domain (which is termed ELD) can comprise Q486E, I499L, N496D mutations and the other modified FokI domain (which is termed KKR) can comprise E490K, I538K, H537R mutations.

In exemplary aspects, the effector domain of the fusion protein is a FokI cleavage domain or a modified FokI cleavage domain.

(iii) Additional Optional Domains

In some aspects, the fusion protein further comprises at least one additional domain. Non-limiting examples of suitable additional domains include nuclear localization signals (NLSs), cell-penetrating or translocation domains, and marker domains.

In certain aspects, the fusion protein can comprise at least one nuclear localization signal. In general, an NLS comprises a stretch of basic amino acids. Nuclear localization signals are known in the art (see, e.g., Lange et al., J. Biol. Chem., 2007, 282:5101-5105). For example, in one embodiment, the NLS may be monopartite sequence such as PKKKRKV (SEQ ID NO:4) or PKKKRRV (SEQ ID NO:5). In another embodiment, the NLS may be a bipartite sequence. In still another embodiment, the NLS may be KRPAATKKAGQAKKKK (SEQ ID NO:6). The NLS may be located at the N-terminus, the C-terminal, or in an internal location of the fusion protein.

In some aspects, the fusion protein can comprise at least one cell-penetrating domain. In one embodiment, the cell-penetrating domain may be a cell-penetrating peptide sequence derived from the HIV-1 TAT protein. As an example, the TAT cell-penetrating sequence may be GRKKRRQRRRPPQPKKKRKV (SEQ ID NO:7). In another embodiment, the cell-penetrating domain may be TLM (PLSSIFSRIGDPPKKKRKV; SEQ ID NO:8), a cell-penetrating peptide sequence derived from the human hepatitis B virus. In still another embodiment, the cell-penetrating domain may be MPG (GALFLGWLGAAGSTMGAPKKKRKV; SEQ ID NO:9 or GALFLGFLGAAGSTMGAWSQPKKKRKV; SEQ ID NO:10). In additional aspects, the cell-penetrating domain may be Pep-1 (KETWWETWWTEWSQPKKKRKV; SEQ ID NO:11), VP22, a cell penetrating peptide from Herpes simplex virus, or a polyarginine peptide sequence. The cell-penetrating domain may be located at the N-terminus, the C-terminal, or in an internal location of the fusion protein.

In still other aspects, the fusion protein can comprise at least one marker domain. Non-limiting examples of marker domains include fluorescent proteins, purification tags, and epitope tags. In some aspects, the marker domain may be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other aspects, the marker domain may be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6xHis, biotin carboxyl carrier protein (BCCP), and calmodulin.

(iv) Fusion Protein Dimers

The present disclosure also contemplates the use of dimers comprising at least one fusion protein as described above. The dimer may be a homodimer or a heterodimer. In some aspects, the heterodimer comprises two different fusion proteins. In other aspects, the heterodimer comprises one fusion protein and an additional protein.

In some aspects, the dimer is a homodimer in which the two fusion protein monomers are identical with respect to the primary amino acid sequence. For example, each fusion protein monomer comprises an identical Cas9 like protein and an identical FokI cleavage domain.

In other aspects, the dimer is a heterodimer of two different fusion proteins. For example, the CRISPR/Cas-like protein of each fusion protein may be derived from a different CRISPR/Cas protein or from an orthologous CRISPR/Cas protein from a different bacterial species. For example, each fusion protein can comprise a Cas9-like protein, which Cas9-like protein is derived from a different bacterial species. In these aspects, each fusion protein would recognize a different target site (i.e., specified by the protospacer and/or PAM sequence). Alternatively, two fusion proteins can have different effector domains. In aspects in which the effector domain is a cleavage domain, each fusion protein can contain a different modified FokI cleavage domain as described above. As will be appreciated by those skilled in the art, the two fusion proteins forming a heterodimer can differ in both the CRISPR/Cas-like protein domain and the effector domain.

Alternatively, the heterodimer may comprise one fusion protein and an additional protein. For example, the additional protein may be a zinc finger nuclease. A zinc finger nuclease comprises a zinc finger DNA binding domain and a cleavage domain. A zinc finger recognizes and binds three (3) nucleotides. A zinc finger DNA binding domain can comprise from about three zinc fingers to about seven zinc fingers. The zinc finger DNA binding domain may be derived from a naturally occurring protein or it may be engineered. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. The cleavage domain of the zinc finger nuclease may be any cleavage domain detailed above in section (I)(c)(ii). In exemplary aspects, the cleavage domain of the zinc finger nuclease is a FokI cleavage domain or a modified FokI cleavage domain. Such a zinc finger nuclease will dimerize with a fusion protein comprising a FokI cleavage domain or a modified FokI cleavage domain. The zinc finger nuclease may comprise at least one additional domain chosen from nuclear localization signals (NLSs), cell-penetrating or translocation domains. Examples of suitable additional domains are detailed above.

### II. Cells

Another aspect of the disclosure provides cells comprising at least one exogenous sequence located in genomic DNA within or proximal to a particular genomic locus. The exogenous sequence is described in section (I) above and comprises the recognition sequence(s) for at least one polynucleotide modification enzyme. In general, the exogenous nucleic acid sequence is stably integrated into the genome, i.e., such that the cell progeny also include chromosomal copies of the exogenous nucleic acid sequence. Transfection and culture protocols intended to yield stable integration are well known in the art, and one of ordinary skill in the art can readily assess whether stable integration has occurred.

The exogenous nucleic acid sequence comprising the recognition sequence(s) for at least one polynucleotide modification enzyme may be located within or proximal to a genomic locus such as the non-limiting examples listed in Table 2, or a homolog, ortholog, or paralog of a genomic locus listed in Table 2. In some embodiments, the genomic locus is associated with high levels of gene expression. An exogenous nucleic acid sequence of the present disclosure may be integrated into or proximal to any accessible genomic locus by any suitable targeting endonuclease as described herein. In certain embodiments, chosen genomic loci are known or unknown “hot” spots or “safe-harbor” spots for recombinant gene expression. Such sites are recognized as regions in the genome that are known to be transcriptionally active and resistant to gene silencing mechanisms to allow for stable gene expression. In some embodiments, an exogenous nucleic acid sequence of the present disclosure may be integrated into a genomic locus identified in Table 2. In other embodiments, an exogenous nucleic acid sequence of the present disclosure may be integrated proximal to a genomic locus identified in Table 2.

Additionally, if multiple landing pads are inserted, each may be located at or near a genomic locus listed in Table 2. For example, an exogenous nucleic acid sequence containing a recognition sequence(s) for at least one polynucleotide modification enzyme may be integrated into two, three, four, five, six, seven, eight, nine, or ten or more genomic locations. As noted herein, multiple copies of the same exogenous nucleic acid sequence may be inserted, or a variety of different exogenous nucleic acid sequences may be inserted.

TABLE 2
Genomic loci in CHO cells

| Gene ID | Protein RefSeq | GeneRefSeq | Base pair |
|---|---|---|---|
| | | NW_003618207.1 | 5366-20679 |
| Rosa26 | | NW_003613637.1 | |
| NEU3 | NP_001231029.1 | NW_003630029.1 | |
| FTH1 | XM_003513182.1 | NW_003615769.1 | |
| ACTB | NM_001244575 | NW_003613618.1 | |
| VEZT | XM_003501431.1 | NW_003613849.1 | |
| CLTA | XM_003513043.1 | NW_003615710.1 | |
| AP1B1 | XM_003505330.1 | NW_003614142.1 | |
| ACTR5 | XM_003497123.1 | NW_003613641.1 | |
| AP3D1 | XM_003502583.1 | NW_003613904.1 | |
| BCS1L | XM_003507606.1 | NW_003614410.1 | |
| COG1 | XM_00345685.1 | NW_003613598.1 | |
| EFTUD2 | XM_003504507.1 | NW_003614071.1 | |
| EIF3I | XM_003500637.1 | NW_003613801.1 | |
| EIF4E2 | XM_003513316.1 | NW_003615857.1 | |
| HIRIP3 | XM_003510112.1 | NW_003614830.1 | |
| NAP1L1 | XM_003506583.1 | NW_003614260.1 | |
| PABPN1 | XM_003506130.1 | NW_003614213.1 | |
| RNF214 | XM_003511972.1 | NW_003615296.1 | |
| TMEM106B | XM_003496525.1 | NW_003613625.1 | |
| ITGA4 | XM_003502135.1 | NW_003613879.1 | |
| UBE2K | XM_003512233.1 | NW_003615402.1 | |
| GNB2L1 | XM_003504042.1 | NW_003614027.1 | |
| ENO1 | XM_003512016.1 | NW_003615313.1 | |
| PSAP | XM_003509296.1 | NW_003614681.1 | |
| MOBKL1B | XM_003510034.1 | NW_003614815.1 | |
| Hypothetical protein | XM_003512615.1 | NW_003615517.1 | |
| LOC100766349 | | | |
| Clone #89 Site 1 | | NW_003617688.1 | 9001-12160 |
| Clone #89 Site 2 | | NW_003615226.1 | 212627-216695 |
| gi\|344162594\|gb\|JH002471.1\| | | NW_003616050.1 | 123022-127022 |
| gi\|344163378\|gb\|JH001687.1\| | | NW_003615266.1 | 282561-286561 |
| gi\|344163843\|gb\|JH001222.1\| | | NW_003614801.1 | 478205-482205 |
| gi\|344164024\|gb\|JH001041.1\| | | NW_003614620.1 | 418875-422875 |
| gj\|344164368\|gb\|JH000697.1\| | | NW_003614276.1 | 423434-427434 |
| | | | and |
| | | | 717777-721777 |
| gj\|344164756\|gb\|JH000309.1\| | | NW_003613888.1 | 528871-532871 |
| gj\|344164561\|gb\|JH000504.1\| | | NW_003614083.1 | 676456-680456 |
| gi\|344164986\|gb\|JH000079.1\| | | NW_003613658.1 | 1320022-324022 |

Cells may be any suitable eukaryotic cell. In exemplary embodiments, the cell is a Chinese Hamster Ovary (CHO) cell, such as cells from the CHO-K1 line or any other suitable cell line. While CHO cells may be the cell of choice, a variety of other cells may also be employed. In general, the cell will be a eukaryotic cell or a single cell eukaryotic organism.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines, in addition to CHO cells, include monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, human U2-OS osteosarcoma cells, human A549 cells, human K562 cells, human HEK293 cells, human HEK293T cells, human HCT116 cells, human MCF-7 cells, and TRI cells. For an extensive list of mammalian cell lines, those of ordinary skill in the art may refer to the American Type Culture Collection catalog (ATCC®, Manassas, Va.). In particular, cell lines useful in recombinant protein production and biopharmaceutical production can be used, for example, CHO cells, mouse myeloma cells (NS0), HEK293 and HEK293T.

In other embodiments, the cell may be a cultured cell, a primary cell, or an immortal cell. Suitable cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

In certain other embodiments, the cell may be an embryo. In some embodiments, the embryo may be a one-cell embryo. The embryo may be a vertebrate or an invertebrate. Suitable vertebrates include mammals, birds, reptiles, amphibians, and fish. Examples of suitable mammals include without limit rodents, companion animals, livestock, and non-primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable non-primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila, mosquitoes, and silkworm.

### III. Methods of Preparing Cells Comprising the Exogenous Sequence

The cells described above may be prepared using any suitable method known to one of ordinary skill in the art. However, in some aspects, a method of preparing a cell comprising a landing pad comprising at least one recognition sequence for a polynucleotide modification enzyme as disclosed herein comprises the steps of (a) introducing into the cell at least one targeting endonuclease (or nucleic acid encoding the targeting endonuclease) targeted to a sequence within or proximal to a genomic locus listed in Table 2; (b) introducing into the cell at least one donor polynucleotide comprising an exogenous nucleic acid comprising at least one recognition sequence for a polynucleotide modification enzyme, a first upstream flanking sequence, and a first downstream flanking sequence, wherein the upstream and downstream sequences have substantial sequence identity with either side of the targeted genomic locus of step (a); and (c) maintaining the cell under conditions such that the targeting endonuclease introduces a double-stranded break at the targeted genomic locus and the double-stranded break is repaired by a homology-directed process such that the exogenous nucleic acid is integrated into the targeted site within or proximal to the genomic locus. Steps (a) and (b) can be performed simultaneously or sequentially; that is, the targeting endonuclease and the donor polynucleotide comprising an exogenous nucleic acid comprising at least one recognition sequence for a polynucleotide modification enzyme and can be administered to the cell at the same time or can be administered in separate steps.

In another aspect, the cell described above may be prepared by (a) introducing into the cell at least one targeting endonuclease (or nucleic acid encoding the targeting endonuclease) targeted to a sequence within or proximal to a genomic locus listed in Table 2; (b) introducing into the cell at least one donor polynucleotide comprising the exogenous nucleic acid sequence comprising at least one recognition sequence for a polynucleotide modification enzyme, a first upstream flanking sequence, and a first downstream flanking sequence, wherein the upstream and downstream sequences comprise the recognition sequence of the targeting endonuclease of step (a); and (c) maintaining the cell under conditions such that the targeting endonuclease introduces a double stranded break in the targeted chromosomal sequence and introduces double stranded breaks in the donor polynucleotide such that the donor polynucleotide is linearized, wherein the linearized donor polynucleotide comprising the exogenous sequence is directly ligated to the cleaved chromosomal sequence, such that the exogenous sequence is integrated into the genome of the cell. Steps (a) and (b) can be performed simultaneously or sequentially.

Accordingly, the present disclosure provides a method for preparing a cell comprising at least one exogenous nucleic acid sequence comprising at least one recognition sequence for a polynucleotide modification enzyme, the method comprising (a) introducing into a cell at least one targeting endonuclease (or nucleic acid encoding the targeting endonuclease) that is targeted to a sequence within or proximal to a genomic locus listed in Table 2; (b) introducing into the cell at least one donor polynucleotide comprising the exogenous nucleic acid that is flanked by (i) sequences having substantial sequence identity to the targeted genomic locus or (ii) the recognition sequence of the targeting endonuclease; and (c) maintaining the cell under conditions such that the exogenous nucleic acid is integrated into genome of the cell. Steps (a) and (b) can be performed simultaneously or sequentially.

The donor polynucleotide containing the exogenous sequence comprising the recognition sequence for a polynucleotide modification enzyme can be single stranded or double stranded, linear, or circular. Generally, the donor polynucleotide is DNA. The donor polynucleotide can be a vector. Suitable vectors include plasmid vectors, phagemids, cosmids, artificial/mini-chromosomes, transposons, and viral vectors. The donor polynucleotide can comprise additional transcriptional control sequencer elements, selectable marker sequences, and/or reporter sequences.

As discussed herein, at least one recognition sequence for a polynucleotide modification enzyme provided in the exogenous nucleic acid may preferably comprise a nucleic acid sequence that does not exist endogenously in the genome of the cell. Other additions and variations to the exogenous nucleic acid sequence are also provided in section I above. For example, the exogenous nucleic acid sequence may optionally comprise at least one selectable marker, at least one sequence for a reporter gene, and/or at least one regulatory control element sequence. In addition, the exogenous nucleic acid sequence may comprise multiple copies of a recognition sequence for a polynucleotide modification enzyme, which recognition sequence may be the same or different.

The methods described herein for preparing cells of the disclosure may also be used to prepare cells containing multiple recognition sites simultaneously. In one aspect, the exogenous nucleic acid introduced into the cell further comprises a second recognition sequence for a second polynucleotide modification enzyme, wherein the first recognition sequence and the second recognition sequence are each recognized by a different polynucleotide modification enzyme. Alternatively, or in addition, steps (a) through (c) of the above-described methods may be repeated using a second exogenous nucleic acid comprising a second recognition sequence, a second upstream flanking sequence, and a second downstream flanking sequence, and a second targeting endonuclease targeted to a different genomic locus than that targeted by the first targeting endonuclease. This process can be repeated with additional exogenous nucleic acid sequences. The exogenous nucleic acid may be presented in an additional plasmid or in another suitable format. The targeted locus may be a locus presented in Table 2 above, or may be another suitable locus known to one of ordinary skill in the art. Such steps may be performed sequentially or simultaneously with steps (a)-(c), as deemed most expedient by one of ordinary skill in the art. In any event, the additional recognition sequence can be any recognition sequence as disclosed herein.

A schematic illustration of an exemplary plasmid comprising an exogenous nucleic acid containing at least one recognition sequence for a polynucleotide modification enzyme of the present disclosure is provided at FIG. 1.

In one aspect, the method comprises introducing into the cell a plasmid comprising at least one exogenous nucleic acid. The exogenous nucleic acid comprises a recognition site for a polynucleotide modification enzyme as provided herein. The exogenous sequence in the plasmid is flanked by an upstream sequence and a downstream sequence, wherein the upstream and downstream sequences either have substantial sequence identity with either side of the targeted locus or comprise the recognition site for the targeting endonuclease used.

As discussed, in one embodiment, the recognition site for a polynucleotide modification enzyme in the exogenous nucleic acid is flanked by an upstream sequence and a downstream sequence that share substantial sequence identity with either side of the targeted cleavage site in the chromosomal sequence. In another embodiment, the recognition site for a polynucleotide modification enzyme in the exogenous nucleic acid is flanked by an upstream sequence and a downstream sequence, each of which comprises the recognition sequence of the targeting endonuclease being used to integrate the exogenous nucleic acid into the genome. One of ordinary skill in the art can readily prepare suitable flanking sequences for any of the loci identified in Table 2 based on their publicly available sequences. Likewise, one of ordinary skill in the art can readily prepare suitable flanking sequences based on the known recognition sequence of the targeting endonuclease used in the method.

The upstream and downstream sequences in the donor polynucleotide comprising the exogenous sequence are selected to promote recombination between the targeted chromosomal sequence and the donor polynucleotide (comprising the exogenous sequence). The upstream sequence, as used herein, refers to a nucleic acid sequence that shares substantial sequence identity with the chromosomal sequence immediately upstream of the targeted cleavage site or comprises the recognition sequence of the targeting endonuclease. Similarly, the downstream sequence in this embodiment refers to a nucleic acid sequence that shares substantial sequence identity with the chromosomal sequence immediately downstream of the targeted cleavage site or comprises the recognition sequence of the targeting endonuclease.

As used herein, the phrase “substantial sequence identity” refers to sequences having at least about 75% sequence identity. Thus, the upstream and downstream sequences in the donor polynucleotide comprising the exogenous sequence may have about 75%, 76%_{,} 77%_{,} 78%_{,} 79%_{,} 80%_{,} 81%_{,} 82%_{,} 83%_{,} 84%_{,} 85%, 86%, 87%, 88%, 89%, 90%, 91%, 92%, 93%, 94%, 95%, 96%, 97%, 98%, or 99% sequence identity with chromosomal sequence adjacent (i.e., upstream or downstream) to the targeted cleavage site or the recognition sequence of a targeting endonuclease. In an exemplary embodiment, the upstream and downstream sequences in the donor polynucleotide comprising the exogenous sequence may have about 95% or 100% sequence identity with chromosomal sequences adjacent to the targeted cleavage site or the recognition sequence of a targeting endonuclease.

An upstream or downstream flanking sequence may comprise from about 10 nucleotides to about 2500 nucleotides. In one embodiment, an upstream or downstream sequence may comprise about 20, 30, 40, 50, 60, 70, 80, 90, 100, 125, 150, 175, 200, 300, 400, 500, 600, 700, 800, 900, 1000, 1100, 1200, 1300, 1400, 1500, 1600, 1700, 1800, 1900, or 2000 nucleotides. An exemplary upstream or downstream flanking sequence may comprise from about 20 to about 200 nucleotides, from 25 to about 100 nucleotides, or from about 40 nucleotides to about 60 nucleotides. In certain embodiments, the upstream or downstream flanking sequence may comprise from about 200 to about 500 nucleotides.

The total length of the exogenous nucleic acid comprising the recognition site that is flanked by the upstream and downstream sequences can and will vary. The exogenous nucleic acid may range in length from about 25 nucleotides to about 5,500 nucleotides. In various embodiments, the donor polynucleotide may be about 50, 100, 200, 300, 400, 500, 600, 800, 1000, 1500, 2000, 2500, 3000, 3500, 4000, or 5000 nucleotides in length.

In some embodiments, the exogenous nucleic acid comprising a recognition site for a polynucleotide modification enzyme used in the methods herein may be provided as a double-stranded, single-stranded, linear or circular sequence. For example, the exogenous nucleic acid may be a plasmid, a bacterial artificial chromosome (BAC), a yeast artificial chromosome (YAC), a viral vector, an oligonucleotide, a synthetic polynucleotide, a polynucleotide linearized by digestion, a PCR fragment, a naked nucleic acid, or a nucleic acid complexed with a delivery vehicle such as a liposome or poloxamer. Typically, the exogenous nucleic acid comprising a recognition site for a polynucleotide modification enzyme will be DNA. In some embodiments, the exogenous nucleic acid may further comprise ribonucleotides, nucleotide analogs, or combinations thereof. A nucleotide analog refers to a nucleotide having a modified purine or pyrimidine base, or a nucleotide comprising a modified ribose moiety. Nucleotide analogs also include dideoxy nucleotides, 2′-O-methyl nucleotides, locked nucleic acids (LNA), peptide nucleic acids (PNA), and morpholinos. The nucleotides may be linked by phosphodiester, phosphothioate, phosphoramidite, phosphorodiamidate bonds, or combinations thereof.

The targeting endonuclease (or encoding nucleic acid) and the exogenous nucleic acid comprising a recognition site for a polynucleotide modification enzyme described herein may be introduced into the cell by a variety of means. Suitable delivery means include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the targeting endonuclease sequence and the exogenous nucleic acid may be introduced into a cell by nucleofection. In another embodiment, the targeting endonuclease sequence and the exogenous nucleic acid may be introduced into the cell by microinjection. For example, the targeting endonuclease sequence and the exogenous nucleic acid may be microinjected into the nucleus or the cytoplasm of the cell. Alternatively, the targeting endonuclease sequence and the exogenous nucleic acid may be microinjected into a pronucleus of a one cell embryo.

In embodiments in which more than one exogenous nucleic acid comprising a recognition site for a polynucleotide modification enzyme are introduced into the cell, the molecules may be introduced simultaneously or sequentially. For example, exogenous nucleic acid comprising a recognition site, each recognition site specific for a particular polynucleotide modification enzyme, may be introduced at the same time. Alternatively, each exogenous nucleic acid comprising a recognition site may be introduced sequentially.

The method further comprises maintaining the cell under appropriate conditions such that the double stranded break introduced by the targeting endonuclease is repaired by homologous recombination or direct ligation such that the exogenous nucleic acid comprising the at least one recognition sequence is integrated into the targeted genomic locus.

In general, the cell will be maintained under conditions appropriate for the particular cell. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

In embodiments in which the cell is a one-cell embryo, the embryo may be cultured in vitro (e.g., in cell culture). Typically, the embryo is cultured at an appropriate temperature and in appropriate media with the necessary O₂/CO₂ ratio to allow the repair of the double-stranded break and allow development of the embryo. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo.

In some instances, the embryo also may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo.

Animals comprising the modified chromosomal sequence may be bred to create offspring that are homozygous for the modified chromosomal sequence. Similarly, heterozygous and/or homozygous animals may be crossed with other animals having genotypes of interest.

### IV. Methods of Using Cells Comprising the Exogenous Sequence

The cells described herein containing one or more landing pad sequences, i.e., one or more exogenous sequences comprising at least one recognition sequence for a polynucleotide modification enzyme, can be used for the production of a recombinant protein, for example, a biopharmaceutical protein. Specifically, the recognition sequence(s) in the landing pad can be targeted by the polynucleotide modification enzyme(s) (i.e., a targeting endonuclease and/or a recombinase) for integration of a sequence encoding the protein of interest. There are several advantages to using the methods and cells described herein containing one or more landing pads that can be retargeted for the production of recombinant proteins. First, one can increase the production of the recombinant protein by increasing the efficiency of the targeted integration (incorporation of the desired genetic material) by choosing a stable genomic locus or loci to insert the landing pad sequence(s) (for subsequent retargeting). Use of a highly efficient targeting endonuclease or recombinase to integrate the genetic sequence of interest (i.e., recombinant protein sequence) into a known, stable location in the genome results not only in the efficient integration of the recombinant protein sequence (the genomic locus or loci may be selected to increase the integrating efficiency of the targeting endonuclease or recombinase), but also the continued, stable expression of the protein sequence following integration. Consequently, this leads to increased cell line stability and decreased clone-to-clone and molecule-to molecule (recombinant protein) heterogeneity, resulting in overall decreased cell line development times and increased protein production. Furthermore, using the methods described herein, it is possible to generate cells comprising multiple landing pad sites for targeted integration of multiple copies of the same recombinant protein or integration of more than one different recombinant protein, thereby providing maximal flexibility as to the protein production that can be achieved. In addition, the inclusion of optional sequences, such as selectable markers, reporter sequences, and/or regulatory control element sequences allows one to further customize the bioproduction capability.

Thus, in a further aspect, the cells described herein containing one or more landing pads or exogenous sequence(s) comprising at least one recognition sequence for a polynucleotide modification enzyme may be retargeted for the production of a recombinant protein or proteins of interest, the method comprising (a) introducing into a cell of the present disclosure (a cell comprising an integrated exogenous sequence(s) containing at least one recognition sequence for a polynucleotide modification enzyme) at least one expression construct comprising a sequence encoding a recombinant protein flanked by an upstream flanking sequence and a downstream flanking sequence, wherein the upstream flanking sequence and downstream flanking sequence are substantially identical to the chromosomal sequence flanking the recognition sequence of the targeting endonuclease of step (b); (b) introducing into the cell at least one targeting endonuclease targeted to a specific recognition sequence present in the exogenous sequence(s) integrated in the cell's chromosomal sequence, wherein the targeting endonuclease introduces a double-stranded break at the recognition sequence; and (c) maintaining the cell under conditions such that the double-stranded break is repaired by a homology-directed process such that the sequence encoding the recombinant protein is integrated into the chromosome. The recombinant protein(s) can be expressed from the retargeted cells using standard protein expression procedures and protocols. Steps (a) and (b) can be performed simultaneously or sequentially; that is, the donor polynucleotide comprising at least one expression construct comprising a sequence encoding a recombinant protein and the targeting endonuclease can be administered to the cell at the same time or can be administered in separate steps.

In still another aspect, the cells described herein containing one or more landing pad sequences may be retargeted for the production of recombinant proteins by (a) introducing into a cell comprising an integrated exogenous sequence comprising at least one recognition sequence for a polynucleotide modification enzyme at least one targeting endonuclease targeted to a specific recognition sequence present in the exogenous sequence integrated in the cell's chromosomal sequence; (b) introducing into the cell at least one expression construct comprising a sequence encoding a recombinant protein that is flanked by the recognition sequence of the targeting endonuclease; and (c) maintaining the cell under conditions such that the targeting endonuclease introduces a double stranded break in the targeted recognition sequence in the landing pad and introduces a double stranded break in the expression construct such that the expression construct is linearized, wherein the linearized expression construct is directly ligated to the cleaved recognition sequence such that the sequence encoding the recombinant protein is integrated into the chromosome. The recombinant protein(s) can be expressed from the retargeted cells using standard protein expression procedures and protocols. Steps (a) and (b) can be performed simultaneously or sequentially.

In yet another aspect, the cells described herein comprising one or more landing pads may be retargeted for the production of recombinant proteins by (a) providing a cell comprising at least one integrated exogenous recombinase recognition sequence; (b) introducing into the cell at least one recombinase that recognizes the recombinase recognition sequence integrated in the cell's chromosomal sequence; (c) introducing into the cell at least one expression construct comprising a sequence encoding a recombinant protein that is flanked by the recognition site for the recombinase; (d) maintaining the cell under conditions such that the recombinase exchanges sequence between the expression construct and the chromosomal sequence such that the sequence encoding the recombinant protein is integrated into the chromosome. The recombinant protein(s) can be expressed from the retargeted cells using standard protein expression procedures and protocols. Steps (a) and (b) can be performed simultaneously or sequentially.

In the present methods, the expression construct may vary within the knowledge and capability of one of ordinary skill in the art as described herein. For example, the expression construct may comprise multiple copies of a single recombinant protein. The expression construct may alternatively or additionally comprise sequences encoding at least two different recombinant proteins. The expression construct may comprise at least one selectable marker (discussed below), at least one reporter gene sequence, and/or at least one regulatory sequence element. For example, the sequence encoding the recombinant protein can be operably linked to a suitable promoter control sequence for expression in a eukaryotic cell. The promoter control sequence can be constitutive or regulated (i.e., inducible or tissue-specific). Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Non-limiting examples of suitable inducible promoter control sequences include those regulated by antibiotics (e.g., tetracycline-inducible promoters), and those regulated by metal ions (e.g., metallothionein-1 promoters), steroid hormones, small molecules (e.g., alcohol-regulated promoters), heat shock, and the like. Non-limiting examples of tissue specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. The promoter sequence can be wild type or it can be modified for more efficient or efficacious expression. Other control elements that may be present include additional transcription regulatory and control elements (i.e., partial promoters, promoter traps, start codons, enhancers, introns, insulators, polyA signals, termination signal sequences, and other expression elements) can also be present.

The recombinant protein can be any recombinant protein, including those useful in biotherapeutic and/or diagnostic application, as well as any recombinant protein useful in industrial applications. For example, the recombinant protein can be, without limit, an antibody, a fragment of an antibody, a monoclonal antibody, a humanized antibody, a humanized monoclonal antibody, a chimeric antibody, an IgG molecule, an IgG heavy chain, an IgG light chain, an Fc region, an IgA molecule, an IgD molecule, an IgE molecule, an IgM molecule, Fc fusion proteins, a vaccine, a growth factor, a cytokine, an interferon, an interleukin, a hormone, a clotting (or coagulation) factor, a blood component, an enzyme, a nutraceutical protein, a glycoprotein, a functional fragment or functional variant of any of the forgoing, or a fusion protein comprising any of the foregoing proteins and/or functional fragments or variants thereof. In exemplary embodiments, the recombinant protein is a human or humanized protein.

In some embodiments, the nucleic acid sequence encoding the recombinant protein may be linked to a nucleic acid sequence encoding an amplifiable selectable marker such as hypoxanthine-guanine phosphoribosyltransferase (HPRT), dihydrofolate reductase (DHFR), and/or glutamine synthase (GS).

In other embodiments, the nucleic acid sequence encoding the recombinant protein may be linked to a nucleic acid sequence encoding a reporter protein such as a fluorescent protein (suitable fluorescent proteins are listed above in section I), glutathione-S-transferase (GST), chitin binding protein (CBP), maltose binding protein, beta-galactosidase, thioredoxin (TRX), biotin carboxyl carrier protein (BCCP), or calmodulin.

### V. Kits

A further aspect of the present disclosure encompasses kits for expression of a recombinant protein of interest. The kits include a cell line comprising at least one exogenous sequence comprising a recognition site for a polynucleotide modification enzyme as described above, an appropriate polynucleotide modification enzyme corresponding to the recognition site, and a construct for insertion of sequence encoding the recombinant protein of interest, wherein the construct further comprises a pair of flanking sequences corresponding to the recognition site sequence or the genomic DNA flanking the recognition site sequence. The kit also includes instructions for completing targeted integration of a sequence encoding the recombinant protein of interest. In one embodiment, the construct for insertion of sequence encoding the recombinant protein of interest further include sequence for a selectable marker, a reporter gene sequence, and/or a regulatory control element sequence. Thus, the kit provides materials and reagents useful in retargeting cells for expression and production of recombinant proteins as discussed above.

In some aspects, the kit includes a cell line comprising more than one exogenous sequence comprising a recognition site (i.e., resulting in more than one recognition site which sites may be the same or different) as described herein, and the appropriate polynucleotide modification enzyme(s) corresponding to the recognition site(s).

In some aspects, the kits include more than one construct for insertion of sequence encoding a recombinant protein of interest, wherein the constructs further comprise a pair of flanking sequences corresponding to a recognition site sequence and/or the genomic DNA flanking a recognition site sequence.

The cell line may be a CHO cell line cell, provided in a sample including a predetermined volume of viable cells. In some aspects the cells may be frozen.

The kit may further comprise one or more additional reagents useful for practicing the disclosed method for recombinant expression of a protein using targeted integration. A kit generally includes a package with one or more containers holding the reagents, as one or more separate compositions or, optionally, as admixture where the compatibility of the reagents will allow. The kit may also include other material(s), which may be desirable from a user standpoint, such as a buffer(s), a diluent(s), culture medium/media, standard(s), and/or any other material useful in processing or conducting any step of the method detailed above.

The kits provided herein preferably include instructions for expressing recombinant proteins as detailed above in section (I). Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

As used herein, the term “polynucleotide modification enzyme” refers to a targeting endonuclease or a site-specific recombinase. Targeting endonucleases can include zinc finger nucleases (ZFNs), meganucleases, transcription activator-like effector nucleases (TALENs), CRIPSR/Cas-like endonucleases, I-Tevl nucleases or related monomeric hybrids, and artificial targeted DNA double strand break inducing agents. Site-specific recombinases can include lambda integrase, Cre recombinase, FLP recombinase, gamma-delta resolvase, Tn3 resolvase, ΦC31 integrase, Bxb1-integrase, and R4 integrase.

The terms “polypeptide” and “protein” are used interchangeably to refer to a polymer of amino acid residues.

As used herein, the term “proximal” means a location near a genomic locus. A proximal location may refer to a location within a predetermined number of nucleotides, i.e., about 10, about 20, about 50, about 100, about 200 nucleotides, or larger distances including 5 kb, 50 kb, or 500 kb and intervening values. Alternatively, an insertion may be proximal to a particular genomic locus if it is relatively closer to one identified locus than to another identified locus, i.e., intergenic sequences.

The term “recognition site,” as used herein, refers to a nucleic acid sequence that is recognized and bound by a polynucleotide modification enzyme, provided sufficient conditions for binding exist. The polynucleotide modification enzyme may be a targeting endonuclease that binds and cleaves the recognition site. Alternatively, the polynucleotide modification enzyme may be a recombinase that mediates exchange between sequences containing the recognition site.

The terms “upstream” and “downstream” refer to locations in a nucleic acid sequence relative to a fixed position. Upstream refers to the region that is 5′ (i.e., near the 5′ end of the strand) to the position and downstream refers to the region that is 3′ (i.e., near the 3′ end of the strand) to the position.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Having described the invention in detail, it will be apparent that modifications and variations are possible without departing from the scope of the invention defined in the appended claims. Moreover, any of the above-listed embodiments or iterations can be combined in any combination.

### EXAMPLES

### Example 1

### Insertion of a ZFN Recognition Landing Pad

ZFN pairs were designed to target Refseq ID NW_003618207.1 at base pairs 12931-12970, Rosa26, and Neu3. ZFNs targeting Refseq ID NW_003618207.1 base pairs 12931-12970, Rosa26, or Neu3 were individually transfected into a suspension adapted CHO K1 cell line. Three days post transfection, ZFN cutting efficiency at the NW_003618207.1, Rosa26, and Neu3 sites in the transfected pool was assessed by the CEL-I Surveyor Mutation Detection Assay or by direct sequencing of InDels (insertions/deletions). When ZFN activity was calculated by direct sequencing of InDels, at least 40 PCR amplicons from each individual site were used in the analysis. The ZFN activity was estimated to be approximately 16%, 31% and 41% at the endogenous CHO site NW_003618207.1, Rosa26, and Neu3 sites, respectively.

Following ZFN validation, a landing pad comprising the recognition sequence for the hAAVS1 ZFN pair was introduced at these three different sites in the CHO genome: Refseq ID NW_003618207.1, Rosa26, and Neu3. A donor plasmid was constructed containing the AAVS1 ZFN recognition sequence flanked by 5′ and 3′ homology arms to Refseq ID NW_003618207.1, Rosa26 and Neu3 sequence, as shown in FIG. 1.

The plasmid donor, as depicted in FIG. 1, was cotransfected with the ZFNs targeting either Refseq ID NW_003618207.1 base pairs 12931-12970, Rosa26, or Neu3 into a suspension adapted CHO K1 cell line. Three days post transfection, the ZFN cutting efficiency at each of the NW_003618207.1, Rosa26, and Neu3 sites in the transfected pool was confirmed by the CEL-I Surveyor Mutation Detection Assay.

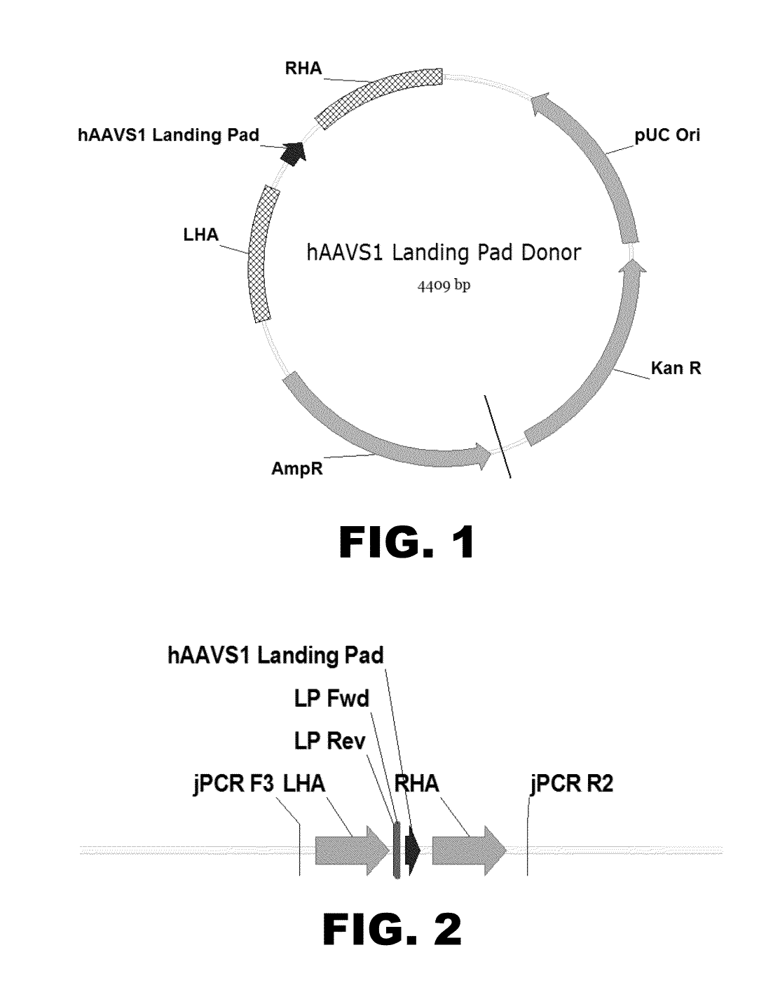

Following the positive CEL-I results, a junction PCR was performed to determine whether targeted integration of the AAVS1 landing pad into the three specified loci had taken place in the transfected pools. The junction PCR was performed with a primer homologous to the CHO genomic DNA just outside of the left (5′) homology arm (“LHA”) or right (3′) homology arm (“RHA”) and a complementary primer homologous to the AAVS1 landing pad, as shown in FIG. 2. A positive PCR product indicated that ZFN-mediated targeted integration (TI) events were present in the transfected pools for each of the loci.

### Example 2

### Activity of ZFN Recognition Landing Pad

The junction PCR positive transfected pools prepared in Example 1 were single cell cloned by limiting dilution cloning. Single cell clones were screened for integration of the landing pad at NW_003618207.1, Rosa26, and Neu3 by junction PCR as described in Example1. Positive clones were scaled up and analyzed.

Clones exhibiting the human AAVS1 landing pad integrated on both alleles at the Refseq ID NW_003618207.1 and Rosa26 loci were isolated and scaled up. Clones exhibiting the AAVS1 landing pad on a single allele at the Neu3 locus were isolated and scaled up. The AAVS1 TI clones were then individually transfected with the human AAVS1 ZFN pair. Three days after transfection, a CEL-I assay or PCR and direct sequencing of InDels was performed at the hAAVS landing pad in the TI clones described above to evaluate AAVS1 ZFN cutting efficiencies in the exogenous landing pad. Forward and reverse primers flanking the AAVS1 ZFN recognition sequence integrated at the three loci (jPCR F3 and R2, as depicted in FIG. 2). The PCR products were sequenced directly or treated with the CEL-I nuclease and analyzed by gel electrophoresis.

Results at the Refseq ID NW_003618207.1 locus demonstrated an average hAAVS1ZFN cutting efficiency of 52% when directly sequencing PCR products. Clones prepared exhibiting the landing pad at the Rosa26 locus demonstrated an average hAAVS1 ZFN cutting efficiency of 18% when using the Cell assay. Clones prepared exhibiting the landing pad at the Neu3 locus demonstrated an average hAAVS1 ZFN cutting efficiency of 16% by directly sequencing PCR products. Adverse phenotypic changes in cell growth and viability were observed in clones containing the landing pad integrated at the Neu3 locus, which may account for the lower efficiency when compared to Rosa26 and Refseq ID NW_003618207.1.

These results demonstrate that an exogenous ZFN recognition sequence can be integrated into the CHO genome at precise locations to generate an engineered landing pad.

### Example 3

### Integration of Recombinant Protein at a ZFN Recognition Landing Pad

A CHO genomic locus for insertion can be determined based on desired expression characteristics and/or ease of integration, such as Refseq ID NW_003618207.1. Targeting endonucleases, such as ZFNs, can be selected or designed based upon the selected genomic locus. As described in Examples 1 and 2 a plasmid can be prepared including a suitable landing pad containing one or more recognition sequences, a reporter and/or selection marker, and one or more regulatory elements. The plasmid can be inserted into a CHO cell along with the targeting endonucleases, and integration of the landing pad can be confirmed using methods such as PCR, sequencing, or Southern blots.

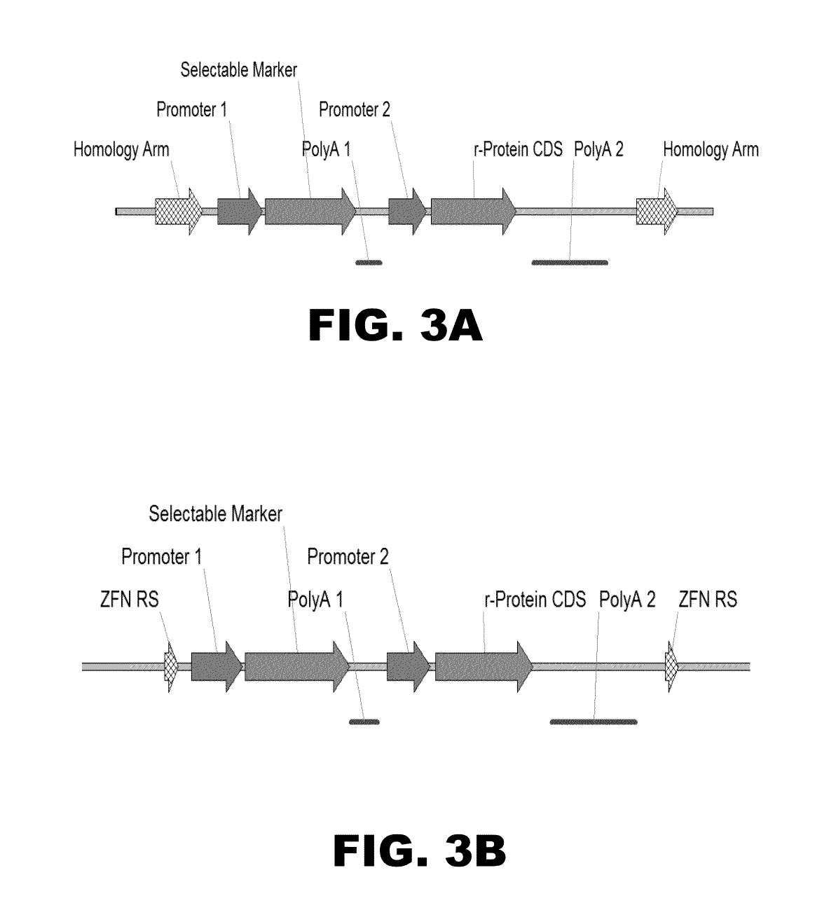

Recombinant protein expression constructs can be then prepared for targeted integration at the landing pad site. The sequence desired for targeted integration (the “payload”) can include two or more independent expression cassettes, one or two for the recombinant protein(s) of interest, such as an IgG heavy chain and/or an IgG light chain, and another for a selectable marker. The payload can be flanked by 5′ and 3′ homology arms to allow for integration by a homology-directed process using a targeting endonuclease (e.g., a pair of ZFNs). A schematic representation is provided at FIG. 3A. Alternatively, the payload can be flanked by targeting endonuclease recognition sequences (i.e., ZFN recognition sequences), or site-specific recombinase recognition sequences, to allow for targeted integration of the payload via direct ligation of cohesive sticky ends or recombinase-mediated cassette exchange (RMCE) respectively. A schematic representation is provided at FIG. 3B. The cells then can be screened to confirm that integration occurred at the targeted site and not randomly.

Results of these analyses are expected to demonstrate that targeted integration occurs at greater rates than random integration when using available selection methods, and that expression of the recombinant protein is stable, homogenous and provided at suitable levels compared to cells in which the recombinant protein was randomly integrated.

1. An isolated cell comprising at least one exogenous nucleic acid sequence located in genomic DNA within or proximal to at least one genomic locus listed in Table 2, wherein each exogenous nucleic acid sequence comprises at least one recognition sequence for a polynucleotide modification enzyme.

2. The isolated cell of claim 1, wherein the cell is a CHO cell.

3. The isolated cell of claim 1 or 2, wherein the at least one recognition sequence comprises a nucleic acid sequence that does not exist endogenously in the genome of the cell.

4. The isolated cell of claim 1, wherein the polynucleotide modification enzyme is selected from the group consisting of a targeting endonuclease, a site-specific recombinase, and combinations thereof.

5. The isolated cell of claim 4, wherein the targeting endonuclease is selected from the group consisting of zinc finger nuclease (ZFN), meganuclease, transcription activator-like effector nuclease (TALEN), CRIPSR endonuclease, I-Tevl nuclease or related monomeric hybrid, and artificial targeted DNA double strand break inducing agent.

6. The isolated cell of claim 4, wherein the site-specific recombinase is selected from the group consisting of lambda integrase, Cre recombinase, FLP recombinase, gamma-delta resolvase, Tn3 resolvase, ΦC31 integrase, Bxb1-integrase, and R4 integrase.

7. The isolated cell of claim 1, wherein a first recognition sequence is recognized by a first ZFN pair.

8. The isolated cell of claim 7, wherein a second recognition sequence is recognized by a second ZFN pair that differs from the first ZFN pair.

9. The isolated cell of claim 7, wherein the first and the second ZFN pair are selected from the group consisting of hSIRT, hRSK4, and hAAVS1.

10. The isolated cell of claim 1, wherein the exogenous nucleic acid sequence further comprises at least one selectable marker sequence, at least one reporter sequence, at least one regulatory control sequence element, or combinations thereof.

11. A method for preparing a cell comprising at least one exogenous nucleic acid sequence comprising at least one recognition sequence for a polynucleotide modification enzyme, the method comprising:
a) introducing into a cell at least one targeting endonuclease that is targeted to a sequence within or proximal to a genomic locus listed in Table 2;
b) introducing into the cell at least one donor polynucleotide comprising the exogenous nucleic acid that is flanked by (i) sequences having substantial sequence identity to the targeted genomic locus or (ii) the recognition sequence of the targeting endonuclease; and
c) maintaining the cell under conditions such that the exogenous nucleic acid is integrated into the genome of the cell.

12. The method of claim 11, wherein the cell is a CHO cell.

13. The method of claim 11 or 12, wherein the exogenous nucleic acid is integrated into the genome by a homology-directed process.

14. The method of claim 11, wherein the exogenous nucleic acid is integrated into the genome by a direct ligation process.

15. The method of claim 11, wherein the targeting endonuclease is selected from the group consisting of zinc finger nuclease (ZFN), meganuclease, transcription activator-like effector nuclease (TALEN), CRIPSR endonuclease, I-Tevl nuclease or related monomeric hybrids, and artificial targeted DNA double strand break inducing agent.

16. A method for retargeting a cell for the production of at least one recombinant protein, the method comprising:
a) providing a cell comprising at least one exogenous recognition sequence for a polynucleotide modification enzyme located within or proximal to at least one genomic locus listed in Table 2;
b) introducing into the cell (i) at least one expression construct comprising a sequence encoding a recombinant protein that is flanked by first and second sequences, and (ii) at least one polynucleotide modification enzyme that recognizes the at least one exogenous recognition sequence in the cell; and
c) maintaining the cell under conditions such that the sequence encoding the recombinant protein is integrated into the genome of the cell.

17. The method of claim 16, wherein the cell is a CHO cell.

18. The method of claim 16, wherein the at least one exogenous recognition sequence of the cell is a targeting endonuclease recognition site; the first and second sequences of the expression construct are sequences with substantial sequence identity to chromosomal sequence near the exogenous recognition sequence in the cell; and the at least one polynucleotide modification enzyme is a targeting endonuclease.

19. The method of claim 16, wherein the at least one exogenous recognition sequence of the cell is a targeting endonuclease recognition site; each of the first and second sequences of the expression construct is the recognition sequence of the targeting endonuclease; and the at least one polynucleotide modification enzyme is a targeting endonuclease.

20. The method of claim 18, wherein the targeting endonuclease is selected from the group consisting of zinc finger nuclease (ZFN), meganuclease, transcription activator-like effector nuclease (TALEN), CRIPSR endonuclease, I-Tevl nuclease or related monomeric hybrid, and artificial targeted DNA double strand break inducing agent.

21. The method of claim 16, wherein the at least one exogenous recognition sequence of the cell is a site-specific recombinase recognition site; each of the first and second sequences of the expression construct is the site-specific recombinase recognition sequence; and the at least one polynucleotide modification enzyme is a site-specific recombinase.

22. The method of claim 21, wherein the site-specific recombinase is selected from the group consisting of lambda integrase, Cre recombinase, FLP recombinase, gamma-delta resolvase, Tn3 resolvase, ΦC31 integrase, Bxb1-integrase, and R4 integrase.

23. The method of claim 16, wherein the sequence encoding the recombinant protein is operably linked to at least one expression control sequence.

24. The method of claim 16, wherein the expression construct further comprises at least one selectable marker sequence, at least one reporter sequence, at least one regulatory control sequence element, or combinations thereof.

25. The method of claim 16, wherein the cells are maintained under conditions for expression of the at least one recombinant protein.

26. A kit for retargeting a cell for the production of a recombinant protein, the kit comprising the cell of claim 1, along with a polynucleotide modification enzyme corresponding to the recognition sequence and a construct for insertion of sequence encoding the recombinant protein of interest, wherein the construct further comprises a pair of flanking sequences corresponding to the recognition sequence and/or the genomic DNA flanking the recognition sequence.

27. The kit of claim 26, further comprising instructions for completing targeted integration of the sequence encoding the recombinant protein.

28. The kit of claim 26, wherein the polynucleotide modification enzyme is a targeting endonuclease selected from the group consisting of zinc finger nuclease (ZFN), meganuclease, transcription activator-like effector nuclease (TALEN), CRIPSR endonuclease, I-Tevl nuclease or related monomeric hybrid, and artificial targeted DNA double strand break inducing agent.

29. The kit of claim 26, wherein the polynucleotide modification enzyme is a site-specific recombinase selected from the group consisting of lambda integrase, Cre recombinase, FLP recombinase, gamma-delta resolvase, Tn3 resolvase, ΦC31 integrase, Bxb1-integrase, and R4 integrase.
